(19) United States
(12) Patent Application Publication
(10) Pub. No.: US 2022/0076309 A1
(43) Pub. Date: Mar. 10, 2022

(54) FLAVORING PROCESS

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/237,767

(22) Filed: Apr. 22, 2021

Related U.S. Application Data
(63) Continuation of application No. PCT/IB2019/058952, filed on Oct. 21, 2019.
(60) Provisional application No. 62/749,075, filed on Oct. 22, 2018.

(51) Int. Cl. G06Q 30/06; G06Q 30/02; A24B 15/167; A24D 1/18

(52) U.S. Cl. CPC G06Q 30/0621; A24D 1/18; A24B 15/167; G06Q 30/0201

(57) ABSTRACT

Systems and methods for matching one or more non-cannabis inputs, such as flavorants, with a cannabis input, such as a cannabis extract, in order to obtain a best match to achieve a desired cannabis-infused edible product are described herein. The systems and methods can include determining a specific purpose for which the non-cannabis inputs are required and determine the best non-cannabis input to masking a non-desirable quality of the cannabis product.

### CROSS-REFERENCE TO RELATED APPLICATIONS

The present patent application is a continuation of International Application No. PCT/IB2019/058952 filed Oct. 21, 2019, which claims the priority benefit of U.S. provisional patent application No. 62/749,075 filed Oct. 22, 2018, the disclosures of which are incorporated by reference herein.

### BACKGROUND OF THE INVENTION

### 1. Field of the Disclosure

The present disclosure is generally related to edible cannabis products. More specifically, the present disclosure is related to the flavoring of edible cannabis products based on the composition of phytochemicals within the product.

### 2. Description of Related Art

Cannabis is a genus belonging to the family cannabaceae. There are three common species of cannabis including Cannabis stavia, Cannabis indica, and Cannabis ruderalis. The genus cannabaceae is indigenous to Central Asia and the Indian subcontinent and has a long history of being used for medicinal, therapeutic, and recreational purposes. For example, cannabis is known to be capable of relieving nausea (such as that accompanying chemotherapy), pain, vomiting, spasticity in multiple sclerosis, and increase hunger in anorexia. The term cannabis as used herein can refer to a “cannabis biomass” which can encompass the Cannabis sativa plant and variants thereof, including subspecies sativa, indica and ruderalis, cannabis cultivars, and cannabis chemovars (varieties characterised by chemical composition). The term “cannabis biomass” is to be interpreted accordingly as encompassing plant material derived from one or more cannabis plants. Such cannabis biomasses can naturally contain different amounts of the individual cannabinoids.

Each cannabis biomass contains a unique class of terpeno-phenolic compounds known as cannabinoids, or phytocannabinoids. The principle cannabinoids present in a cannabis biomass can include Delta-9-tetrahydrocannabinolic acid (THCA) and cannabidiolic acid (CBDA). THCA does not include psychoactive properties on it's own, but when decarboxylated THCA becomes Delta-9-tetrahydrocannabinol (THC), which is a potent psychoactive cannabinoid. CBDA can be decarboxylated into cannabidiol (CBD), which is a major cannabinoid substituent in hemp cannabis. CBD is a non-psychoactive cannabinoid and is widely known to have therapeutic potential for a variety of medical conditions including, but not limited to, those described above.

Historical delivery methods of cannabinoids have included combustion (such as smoking) of the dried cannabis plant material, or biomass. However, smoking can result in adverse effects on a user's respiratory system due to the production of potentially toxic substances. Moreover, smoking is an inefficient mechanism which delivers a variable mixture of both active and inactive substances, many of which may be undesirable. Common alternative delivery methods, including but not limited to, ingestion, typically require an extraction process to be performed on the cannabis biomass to remove the desired components. Such ingestible cannabis items can include, but are not limited to, concentrates, extracts, and cannabis oils.

A cannabis edible, also known as a cannabis-infused food, edible cannabis product, or simply an “edible,” can refer to a food product that contains cannabinoids, such as tetrahydrocannabinol (THC) and cannabidiol (CBD). Although an edible may generally refer to either a food or a drink, a cannabis-infused drink may be referred to specifically as a liquid edible or a drinkable. For the purposes of this disclosure, the term “food product” can encompass any form of cannabis edible including liquid edibles. Most edibles contain a significant amount of THC, which can induce a wide range of effects, including, but not limited to, relaxation, euphoria, increased appetite, fatigue, and anxiety. THC-dominant edibles are consumed for recreational and medical purposes. In the alternative, some edibles can only contain a negligible amount of THC, and are instead dominant in other cannabinoids, most commonly CBD. Such CBD edibles are primarily used for medical purposes. Foods and beverages made from such non-psychoactive cannabis products are sometimes known as hemp foods.

THCA may degrade into THC, which may be degrade into cannabinol over time. THCA can be rapidly, albeit not completely in many instances, decarboxylated when heated. Comparing effects of eating cannabis products and smoking them is difficult because there are large margins of error due to variability in how different people smoke, with the number, duration, and spacing of puffs, the hold time, and the volume of the person's lungs, all of which may result in different types and extent of the effects of the dosage.

With regard to eating, different vehicles in which cannabinoids are dissolved for oral consumption can affect the availability of the cannabinoids to be absorbed. Additionally, different people can metabolize the same products differently. Generally, however, because oral cannabis doses are processed by the digestive system and the liver before entering the bloodstream, ingested cannabinoids may be are absorbed more slowly, have delayed and lower peak concentrations, and are cleared through the user's system more slowly in comparison to the inhalation of the same in an aerosol such as that which is formed when cannabis is burnt.

Oral administration of cannabinoids generally leads to two concentration peaks, due to enterohepatic circulation. Consuming THC through ingestion results in absorption through the liver and, through metabolic processes, the conversion of a significant proportion of it into 11-hydroxy-THC, which is more potent than THC.

Cannabis-infused products can have an “off” flavor; as such flavoring is typically added to the cannabis-infused products in order to mask the flavor of the cannabis concentrate. However, some products can end up having too much flavoring resulting in a poor tasting edible.

### SUMMARY OF THE CLAIMED INVENTION

Examples of the present disclosure provide systems and methods for determining a match between a cannabis input and one or more non-cannabis inputs. In particular, a system for determining the best non-cannabis inputs to mask a non-desirable flavor of a cannabis input can include a cannabis subsystem, a non-cannabis subsystem, a desired outcome subsystem, and a matching analytics subsystem communicably coupled with one another via a communication network. The matching analytics subsystem having an Artificial Intelligence (“AI”) or machine learning algorithm module operable to compare the flavor profile of a cannabis input with one or more non-cannabis inputs in order determine the best match to achieve a desired cannabis-infused edible product.

In addition to improving the flavor of resulting cannabis-infused edible products, such systems and methods can further provide secondary matches which can be substituted for the original best match.

### BRIEF DESCRIPTIONS OF THE DRAWINGS

FIG. 1 illustrates an exemplary network environment in which a system for flavoring a cannabis-infused food product may be implemented.

FIG. 2 is a flowchart illustrating an exemplary method for using an AI algorithm module.

FIG. 3 is a flowchart illustrating an exemplary method for using a data collection module.

FIG. 4 is a flowchart illustrating an exemplary method for using an outcome module.

FIG. 5 is a flowchart illustrating an exemplary method for using an outcome database.

FIG. 6 is a flowchart illustrating an exemplary method for using a cannabis module.

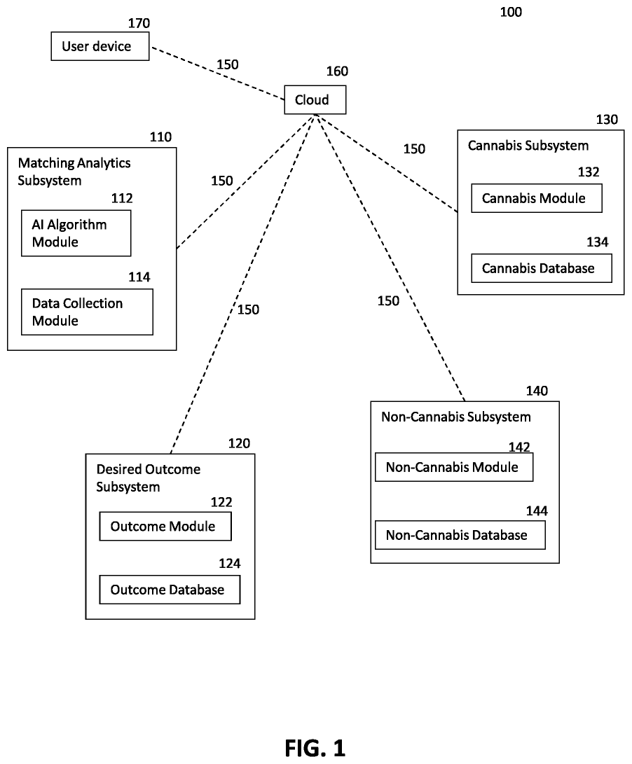

FIG. 1 is a flowchart illustrating an exemplary method for using a cannabis database.

FIG. 8 is a flowchart illustrating an exemplary method for using a non-cannabis module.

FIG. 9 is a flowchart illustrating an exemplary method for using a non-cannabis database.

### DETAILED DESCRIPTION

As described above, the inclusion of cannabinoids in a food product can change the taste of the food product. In at least some examples, the cannabinoids can cause an “off” taste in the edible. As such, the cannabis industry represents an enormous opportunity for flavoring suppliers. While cannabis edibles might seem like the most obvious market for flavorists to exploit, cannabis concentrates, and in particular those forms used for example in vaping liquids and oils, can also potentially be of interest because flavorings can form a large part of the cannabis concentrates experience. Concentrates can also appear in cannabis edibles, although standalone concentrate products are a likely avenue for flavorists looking to enter the market.

Edibles are often flavored to mask the flavor of cannabis, whereas cannabis vaping liquid products tend to highlight the flavor of cannabis. Common terpenes like limonene, which can be found in citrus, or beta-myrcene, which can be found in hops, are responsible for the distinct flavors that differentiate various cultivars, or strains, of cannabis. The distinctions between the various cultivars can be even more pronounced when it comes to concentrates, such as those extracted from the cannabis biomass. This is because the extraction technology used has reached the point where individual molecules of cannabis can be separated and recombined, creating custom blends of cannabinoids including, but not limited to, THC, CBD, and terpenes.

The present disclosure is generally related to creating the right combination of additives, such as additives, which can enhance the product experience to mask, add to, or enhance the cannabis flavor impact to food products. More specifically, the present disclosure addresses how to match non-cannabis flavors with cannabis extracts to mask non-desirable flavors. Such non-desirable flavors can include, but are not limited to, any organoleptic element such as sour flavors, salty flavors, or umami flavors to reduce bitterness. A need exists to find a flavoring methodology specific to the addition of marijuana concentrates. Moreover, the present disclosure provides a method to mask a specific non-desirable quality in the extract for a specific purpose, which may be, for example, manufacturing an edible food or beverage product. An extract with added characteristics including, but not limited to, flavoring, coloring, and diluent, can be crated for a specific purpose, such masking flavor or enhancement of a flavor or the elimination of certain flavors.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

FIG. 1 illustrates an exemplary network environment in which a system 100 for flavoring a cannabis-infused food product may be implemented. Such system 100 may be used in determining the appropriate mixture of non-cannabis flavors to mask the flavor of cannabis in an edible food product. The system 100 can comprise a matching analytics subsystem 110 operable to execute an AI algorithm module 112 to match cannabis and non-cannabis materials to achieve a desired outcome. The AI algorithm module 112 can be operable to perform various correlations between data in order to determine if there is a formula for combining cannabis and non-cannabis elements in order to achieve a specific purpose. The system 100 can further include a desired outcome subsystem 120, a cannabis subsystem 130, and a non-cannabis subsystem 140 communicably coupled with one another via a communications network 150.

The matching analytics 102 can further include a data collection module 114, the data collection module 106 can be operable to collect data from the desired outcome subsystem 110, the cannabis subsystem 130, and the non-cannabis subsystem 140. Data from each of the subsystems can be used by for the AI algorithm module 112 to compare and match correlations between different amounts of cannabis and non-cannabis factors, or elements, to determine the best combination for a specific purpose. For example, the desired outcome subsystem 120 can be operable to determine a desired outcome. In at least one example, the desired outcome is a food product flavor. The desired outcome subsystem 120 can include an outcome module 122 operable to transmit outcome data to the data collection module 114 of the matching analytics subsystem 110. In at least one example, the data outcome module can further be operable to receive potential specified outcome information from a user via a user device 170. An outcome database 124 stored on the desired outcome subsystem 120 can be operable to store various outcome properties and the specific purpose for such outcome properties. In at least one example, the outcome properties and purposes can relate to combining cannabis and non-cannabis factors to create an edible cannabis-infused product.

Specifically, the cannabis subsystem 130 can be operable to determine a flavor profile relating to a cannabis feedstock which can be used in the manufacture of a cannabis-infused a food or beverage product. The cannabis feedstock may be any form of cannabis suitable for manufacturing into an edible cannabis product, including but not limited to cannabis biomass, cannabis extracts and liquid and solid formulations of cannabis extracts. A cannabis module 132 stored on the cannabis subsystem 130 can be operable to transmit cannabis data consisting of cannabis factors to the data collection module of the matching analytics subsystem 110 described above. The cannabis module 132 can further be operable to receive specified cannabis factor data from the user device 170. The cannabis subsystem 130 can further include a cannabis database 134 stored thereon and operable to store various cannabis properties and factors which can be taken into account when combining a cannabis input, such as a cannabis extract or cannabis concentrate, with a non-cannabis input, or element. Similarly, the non-cannabis subsystem 140 can provide data relating to non-cannabis materials which can be in the manufacture of a food or beverage product. The non-cannabis subsystem 140 can have a non-cannabis module 142 stored thereon and operable to transmit non-cannabis data consisting of various non-cannabis factors to the data collection module 114 of the matching analytics subsystem 110. The non-cannabis module 142 can also be operable to receive specified non-cannabis factor data from a user device. The non-cannabis subsystem 140 can have a non-cannabis database 144 stored thereon and operable to store the properties and factors of non-cannabis inputs for use in the manufacture of cannabis-infused products.

The communication network 150 may be inclusive of wired and/or wireless networks. The communication network 150 may be implemented, for example, using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. The communication network 150 can allow ubiquitous access to shared pools of configurable system resources and higher-level services that can be rapidly provisioned with minimal management effort, often over Internet and relies on sharing of resources to achieve coherence and economies of scale, like a public utility, while third-party clouds may enable organizations to focus on their core businesses instead of expending resources on computer infrastructure and maintenance. In at least one example, the matching analytics subsystem 110, the desired outcome subsystem 120, the cannabis subsystem 130, and the non-cannabis subsystem 140 can be accessed by a user via an application on a user device 170. In at least one example, the application can include an API. The API (or application programming interface) is an application-specific interface that can allow users to send and receive information to the various subsystems. The modules, databases, and networks described with respect to FIG. 1 can be stored on, and accessible via, the cloud network 160.

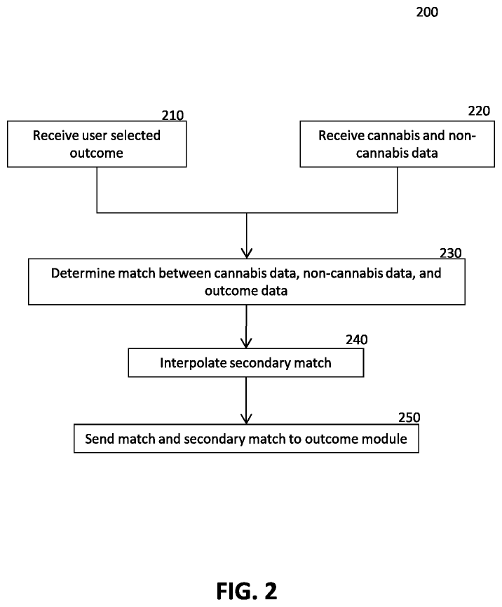

A method 200 for using the AI algorithm module to determine an amount of cannabis and non-cannabis factors to include in a specific cannabis-infused product is illustrated in FIG. 2. In at least one example, the cannabis and non-cannabis factors can correspond to a cannabis input and one or more non-cannabis inputs. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The method 200 can begin at block 210 wherein the AI algorithm module 112 receives outcome data from the data collection module 114 to initiate matching what outcome a user desires. In at least one example, the data can describe a particular flavor desired or final product flavor profile, such as a honey flavored hard candy. The outcome data can include the sweetness (for example perceived sweetness, relative to sucrose), a flavor profile (such as sweet, mild spicy note, floral aroma, etc.), mouthfeel (smooth and rich), and various other desirable elements of taste. At block 220, the AI algorithm module 112 can receive cannabis data and non-cannabis data from the data collection module 114. The cannabis data can include, but is not limited to, a cannabis cultivar and its associated flavor profile, which may be, for example, bitter, floral, citrus, flavor profile of a sweet fruit, a sweet flavored THC edible, and combinations thereof. The cannabis data can be correlated to a specific cannabis input used in the manufacturing process. The non-cannabis data can include, but is not limited to, other ingredients used in the manufacture of food products. In the example described above, the non-cannabis data for a honey flavored hard candy can include an associated flavor profile, which may be, for example, honey, sugar, gelatin, corn syrup, lemon or orange extract, red, yellow, or orange food coloring, and the like. The non-cannabis data can be correlated to one or more non-cannabis inputs for use in the manufacturing process.

At block 230, the AI algorithm module can use the received cannabis data, non-cannabis data, and outcome data to calculate a match. The match calculated can include, for example, a flavor, additive to mitigate a cannabis flavor, a flavor additive to enhance the flavor of other ingredients (for example, a honey flavor additive), or a flavor additive to create a specific outcome not associated with either the cannabis or the non-cannabis ingredients (such as a ‘tropical’ flavor or other novelty flavors not directly associated with any ingredients in the candy. Specifically, the match can be determined by comparing the cannabis data, non-cannabis data, and outcome data to determine a match for the user selected outcome based on a combination of cannabis data and non-cannabis data. In the example provided above, data is compared based on a selected outcome of a honey flavored hard candy THC edible such that the AI algorithm module can match a cannabis input, or cannabis feedstock, having a particular flavor profile and one or more non-cannabis inputs having sweet flavor profiles are determined to match. For example, a specific cannabis feedstock which produces honey-like flavors may be the best match for the honey flavored hard candy THC edible. In at least one example, the cannabis input can be predetermined by a user based on the cannabis feedstock available to the manufacturer, and one or more of the most compatible non-cannabis inputs can be determined based on the AI algorithm module to achieve the desired outcome.

At block 240, a second match can be interpolated by comparing the cannabis data, non-cannabis data, and the matches determined in step 230 to determine at least a singular factor which can be altered in order to achieve the same user selected outcome through a different match. For example, if a sweet THC edible is the desired outcome, a cannabis input, or cannabis feedstock, can be selected, and a sweet flavor, such as strawberry, can be selected. In this example, the singular factor that could be altered is a non-cannabis input such as the strawberry flavor. The factor which can be altered by selecting another flavor similar to strawberry, such as blackberry, blueberry, raspberry, mint, ginger, black pepper, chocolate, citrus, and rhubarb. Finally, at block 250, the match data and secondary match data can be transmitted to the outcome module 122. The transmission can be, for example, the cannabis input, including cannabis feedstock, and the non-cannabis input, such as strawberry match. The transmission can also include the THC amount and blackberry match can be sent to the outcome module 122.

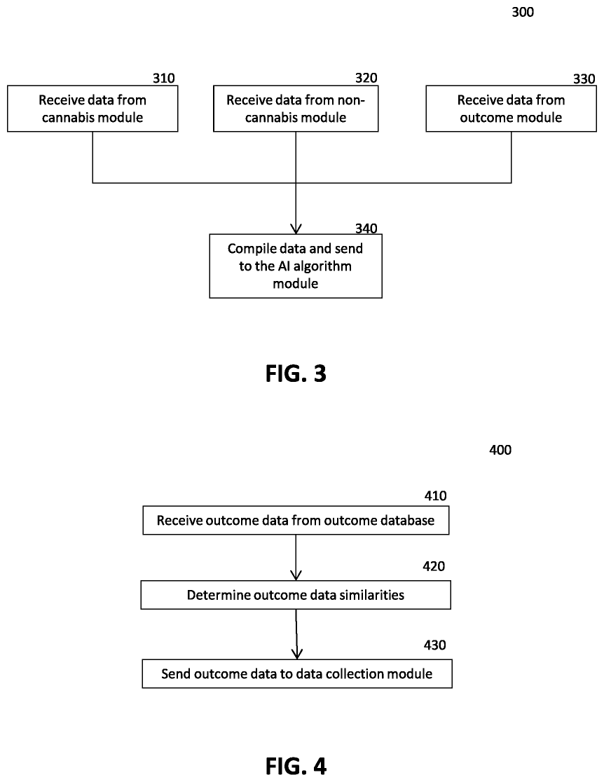

Methods describing the functioning of each of the modules above are explained in further detail below with respect to FIGS. 3 to 9.

For example, the functioning of the data collection module 114 described in FIGS. 1 and 2, is explained with reference to FIG. 3. Specifically, FIG. 3 is a flowchart illustrating an exemplary method 300 for using a data collection module. The method 300 can begin at block 310 where cannabis data relating to various cannabis inputs can include, but is not limited to, cannabis feedstock, flavonoids, and terpenes, is received from the cannabis module 132. The cannabis data can include a flavor profile and cannabinoid (such as THC or CBD) amount for each cannabis feedstock. For example, the cannabis data for a cannabis feedstock named OG Kush Concentrate can include a flavor profile that is described as deep sour-lime and a piney undertone, and a THC concentration of, for example 60%. At block 320, non-cannabis data can be received from the non-cannabis module 142 of the non-cannabis subsystem 140. The non-cannabis data relating to one or more non-cannabis inputs can include, but is not limited to, coloring, flavoring, extract, and dilution. For example, non-cannabis data can be received comprising a flavor profile of strawberry and blackberry. At block 330, outcome data is received from the outcome module 122 of the desired outcome subsystem 120. The outcome data can include, but is not limited to, recipes, formula, and previous solutions to user selected outcomes. For example, the outcome data can include a recipe for a THC edible. In at least one example, the cannabis data relating to a specific cannabis input and the desired outcome can be selected by a user based on the cannabis input available and the product they wish to produce. For example, if an edible cannabis product manufacturer has a specific cannabis concentrate or extract and they intend to produce a batch of honey flavored hard candies, such information can be entered into the data collection module. Finally, at block 340, the cannabis, non-cannabis, and outcome data collected can be compiled and transmitted to the AI algorithm module 112 for determining a match between the received sets of data as described above with respect to FIG. 2.

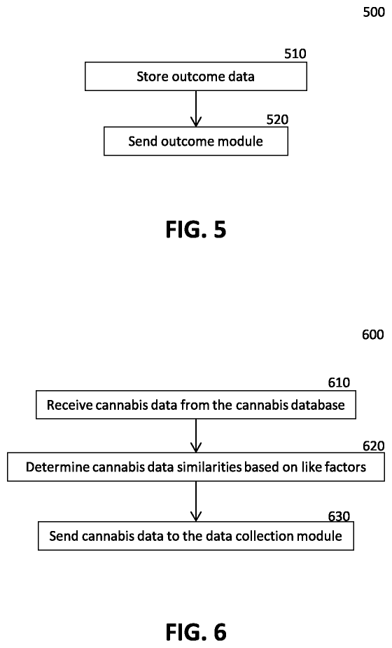

The functioning of the outcome module is explained with reference to FIG. 4, which is a flowchart illustrating an exemplary method 400 for using an outcome module to evaluate outcomes. The method 400 can begin at block 410 where outcome data such as color adjustment, flavor adjustment, and dilution adjustment is received at the outcome module 122 from the outcome database 124. In at least one example, outcome data can be received which describes a honey flavored hard candy, including the sweetness (such perceived sweetness relative to sucrose), flavor profile (such as sweat, mild spicy note, floral aroma, and the like), mouthfeel (such as smooth and rich), as well as other elements of taste. At block 420, the outcome data is compared to determine a like factor, such as a similar color adjustment, flavor adjustment, or dilution adjustment as described in detail with respect to FIG. 2. At block 430 the outcome data such as color adjustment, flavor adjustment, and dilution adjustment are sent from the outcome database 124 to the data collection module 120.

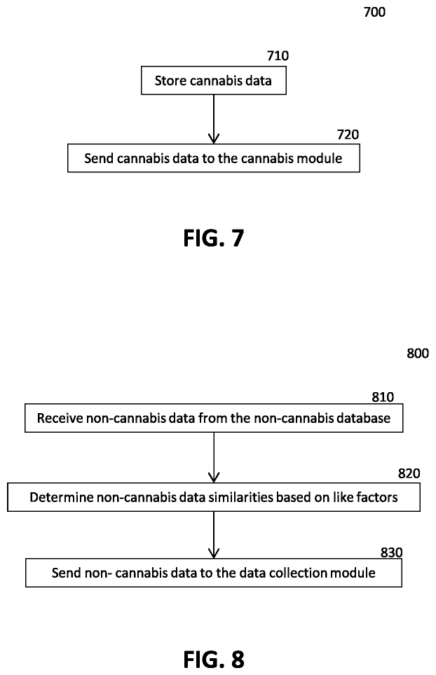

Further functioning of the outcome database is explained with reference to FIG. 5, which is a flowchart illustrating an exemplary method 500 for using an outcome database. The method 500 can begin at block 510 where outcome data such as color adjustment, flavor adjustment, and dilution adjustment can be stored in the outcome database 124. In at least one example, the outcome data stored in the outcome database 124 can be organized based on final outcome. For example, outcome data comprising flavor profile adjustments, such as strawberry and blackberry, for masking cannabis off-notes can be stored together. At block 520, the outcome data such as color adjustment, flavor adjustment, and dilution adjustment are transmitted from the outcome database 124 to the outcome module 122.

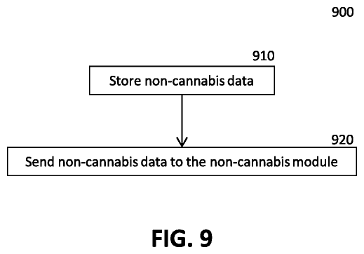

The functioning of the cannabis module is explained with reference to FIG. 6, which is a flowchart illustrating an exemplary method 600 for using a cannabis module. The method 600 can begin at block 610 where the cannabis data, such as cannabis feedstock, flavonoids, and terpenes, can be received at the cannabis module 132 from the cannabis database 134. As described above, the cannabis data can include, but are not limited to, information relating to various cannabis feedstocks including a flavonoid strength and terpene level. At block 620, the cannabis data can then be compared to determine a like factor such as a similar feedstock, flavonoid, or terpenes, as described above. For example, cannabis data comprising various cannabis feedstock flavors which can be compared with another. At block 630, the cannabis data such as cannabis cultivar, flavonoids, and terpenes can be transmitted from the cannabis module 132 to the data collection module 114.

The functioning of the cannabis database is explained with reference to FIG. 7, which is a flowchart illustrating an exemplary method 700 for using a cannabis database. The method 700 can begin at block 710 where the cannabis data relating to cannabis inputs described above is stored in the cannabis database 134. At block 720, the cannabis data can be transmitted from the cannabis database 134 to the cannabis module 132 of the cannabis subsystem 130.

The functioning of the non-cannabis module is explained with reference to FIG. 8, which is a flowchart illustrating an exemplary method 800 for using a non-cannabis module. The method 800 can begin at block 810 where non-cannabis data, relating to one or more cannabis inputs as described above, including, but not limited to, masking flavors, masking colorings, and masking ingredients can be received from the non-cannabis database 114. In at least one example, the non-cannabis flavor profiles can include of each ingredient used in a honey flavored candy, such as sugar, corn syrup, gelatin, and the like. At block 820, the non-cannabis data such as masking flavors, masking colorings, and masking ingredients are compared to determine a like factor such as a masking flavor, masking coloring, or masking ingredient. For example, as described above, comparing a strawberry flavor profile to a blackberry flavor profile. At block 830 the non-cannabis data can then be transmitted from the non-cannabis module 142 to the data collection module 114.

The functioning of the non-cannabis database is explained with reference to FIG. 9, which is a flowchart illustrating an exemplary method 900 for using a non-cannabis database. The method 900 can begin at block 910 where the non-cannabis data relating to non-cannabis inputs described above is stored in the non-cannabis database 144 of the non-cannabis subsystem 140. At block 920, the non-cannabis data can be transmitted from the non-cannabis database 144 to the non-cannabis module 142. The non-cannabis data can then subsequently be transmitted to the data collection module 114 of the matching analytics subsystem 110.

What is claimed is:

1. A method for matching non-cannabis inputs with a cannabis inputs used in the manufacture of an edible cannabis product, the method comprising:
receiving data sent over a communication network, the received data relating to a plurality of cannabis inputs, non-cannabis data relating to one or more non-cannabis inputs, and outcome data relating to a desired outcome;
evaluating the received data to identify one or more matches between the cannabis inputs, the non-cannabis inputs, and the desired outcome;
selecting one of the matches to send to an outcome subsystem; and
transmitting the selected match to the outcome subsystem over the communication network.

2. The method of claim 1, further comprising:
storing the cannabis data in a cannabis database of a cannabis subsystem;
storing the non-cannabis data in a non-cannabis database of a non-cannabis subsystem;
storing the outcome data in an outcome database of an outcome subsystem, wherein receiving the data includes retrieving at least one of the cannabis data from the cannabis database, the non-cannabis data from the non-cannabis database, and the outcome data from the outcome database, and wherein evaluating the received data includes compiling the cannabis data, the non-cannabis data, and the outcome data.

3. The method of claim 1, wherein the cannabis data includes one or more of a cannabis feedstock and a cannabis flavor profile.

4. The method of claim 3, wherein the cannabis feedstock includes one or more of a cannabis biomass, a liquid formulation of cannabis extracts, and a solid formulation of cannabis extracts.

5. The method of claim 1, wherein the non-cannabis data includes one or more of a masking flavor, a masking coloring, and a masking ingredient.

6. The method of claim 1, wherein the outcome data includes one or more of a sweetness, an edible flavor profile, and a mouthfeel.

7. The method of claim 1, wherein identifying the matches further comprises:
comparing the cannabis data to the non-cannabis data to identify one or more possible combinations; and
determining which of the combinations are associated with the desired outcome.

8. The method of claim 1, wherein the selected match includes at least one elected cannabis input and at least one elected non-cannabis input.

9. The method of claim 8, further comprising interpolating a secondary match, wherein the secondary match is also transmitted over the communication network to the outcome sub-system.

10. The method of claim 1, wherein the desired outcome is based on a user selection.

11. A system for matching non-cannabis inputs with a cannabis input used in the manufacture of an edible cannabis product, the system comprising:
a communication network interface that receives data sent over a communication network, the received data relating to a plurality of cannabis inputs, non-cannabis data relating to one or more non-cannabis inputs, and outcome data relating to a desired outcome;
an analytics module stored in memory and executable by a processor to:
evaluate the received data to identify one or more matches between the plurality of cannabis inputs, the one or more non-cannabis inputs, and the desired outcome, and
select one of the matches to send to an outcome subsystem;
wherein the communication interface transmits the selected match to the outcome subsystem over the communication network.

12. The system of claim 11, further comprising:
a cannabis database of a cannabis subsystem that stores the cannabis data.
a non-cannabis database of a non-cannabis subsystem that stores the non-cannabis data;
an outcome database of an outcome subsystem that stores the outcome data, wherein the communication interface receives the data by retrieving at least one of the cannabis data from the cannabis database, the non-cannabis data from the non-cannabis database, and the outcome data from the outcome database, and wherein the analytics module evaluates the received data by compiling the cannabis data, the non-cannabis data, and the outcome data.

13. The system of claim 11, wherein the cannabis data includes one or more of a cannabis feedstock and a cannabis flavor profile, wherein the cannabis feed stock includes one or more of a cannabis biomass, a liquid formulation of cannabis extracts, and a solid formulation of cannabis extracts.

14. The system of claim 11, wherein the non-cannabis data includes one or more of a masking flavor, a masking coloring, and a masking ingredient.

15. The system of claim 11, wherein the outcome data includes one or more of a sweetness, an edible flavor profile, and a mouthfeel.

16. The system of claim 11, wherein the analytics module identifies the matches by:
comparing the cannabis data to the non-cannabis data to identify one or more possible combinations; and
determining which of the combinations are associated with the desired outcome.

17. The system of claim 11, wherein the selected match includes at least one elected cannabis input and at least one elected non-cannabis input.

18. The system of claim 17, wherein the analytics module is further executable to interpolate a secondary match, wherein the communication network interface also transmits the secondary match over the communication network to the outcome sub-system.

19. The system of claim 11, wherein the desired outcome is based on a user selection.

20. A non-transitory, computer-readable storage medium, having embodied thereon a program executable by a processor to perform a method for matching non-cannabis inputs with a cannabis input used in the manufacture of an edible cannabis product, the method comprising:
receiving data sent over a communication network, the received data relating to a plurality of cannabis inputs, non-cannabis data relating to one or more non-cannabis inputs, and outcome data relating to a desired outcome;
evaluating the received data to identify one or more matches between the cannabis inputs, the non-cannabis inputs, and the desired outcome;
selecting one of the matches to send to an outcome subsystem; and
transmitting the selected match to the outcome subsystem over the communication network.
